(19) United States
(12) Patent Application Publication
(10) Pub. No.: US 2014/0275724 A1
(43) Pub. Date: Sep. 18, 2014

(54) PERCUTANEOUS DUAL-LUMEN CANNULA FOR CAVOPULMONARY ASSIST DEVICE

(71) Applicant: W-Z Biotech, LLC, (US)

(72) Inventors: Dongfang Wang, Lexington, KY (US); Joseph B. Zwischenberger, Lexington, KY (US)

(73) Assignee: W-Z BIOTECH, LLC, Lexington, KY (US)

(21) Appl. No.: 13/836,880

(22) Filed: Mar. 15, 2013

(51) Int. Cl. A61M 1/10

(52) U.S. Cl. CPC A61M 1/1086
USPC 600/16

(57) ABSTRACT

A dual-lumen cannula for cavopulmonary assistance includes a body defining an infusing lumen and a draining lumen and a deployable barrier disposed on an exterior surface of the body. Deploying the barrier within a vascular lumen substantially occludes fluid flow therethrough other than in an intravascular zone defined by the deployed barrier. The deployable barrier includes a first deployable wall spaced apart from a second deployable wall. The infusing lumen includes outlets disposed such that on initiation of fluid flow through the infusing lumen, that fluid flow promotes deployment of the first and second deployable walls, which in embodiments are foldable membrane umbrellas or cones having at least one end thereof attached to the cannula body. Percutaneous dual-lumen cannula based cavopulmonary assist devices incorporating the dual lumen cannula and methods for using to support or remedy failing Fontan circulation are described.

### TECHNICAL FIELD

The present invention relates generally to the field of cardiac medical devices and systems. In particular, the invention relates to a dual lumen cannula for use in remediating or alleviating failing circulation subsequent to a Fontan procedure, to cavopulmonary assist devices incorporating the dual lumen cannula, and to methods of use of the dual lumen cannula and cavopulmonary assist devices.

### BACKGROUND OF THE INVENTION

The Fontan or Fontan/Kreutzer procedure is a palliative (not curative) surgical procedure used to ameliorate complex congenital heart defects, especially in the young. Exemplary heart defects addressed by the Fontan procedure include heart valve defects (tricuspid atresia, pulmonary atresia), abnormalities in pumping ability of the heart (hypoplastic left heart syndrome, hypoplastic right heart syndrome), and other complex congenital heart diseases where a bi-ventricular repair is not possible or contra-indicated (double inlet left ventricle, heterotaxy defects, double outlet right ventricle, etc.). The procedure was initially described as a surgical treatment for tricuspid atresia.

In the Fontan procedure, a surgically created junction is provided between the superior and inferior vena cava and the pulmonary artery, and venous blood flow is diverted from the superior and inferior vena cava directly to the pulmonary artery, bypassing the right ventricle of the heart. Following the procedure, oxygen-poor blood from the upper and lower body flows through the lungs without being pumped by the heart. Rather, the blood flow into the lungs is driven only by central venous blood pressure. This corrects hypoxia, and leaves a single heart ventricle responsible only for supplying blood to the body.

However, disadvantages and post-surgical complications are associated with the Fontan procedure. In the short term, pleural effusions (fluid build-up around the lungs) can occur, requiring additional surgical interventions. In the long term, atrial scarring may be associated with atrial flutter and atrial fibrillation, also requiring additional surgical intervention. Other long-term risks may be associated with the procedure, such as protein-losing enteropathy and chronic renal insufficiency, although these latter risks are not yet fully quantified.

Also, a high central venous pressure is required to provide a satisfactory supply of blood to the lungs after the Fontan procedure. Immediately or even 2-5 years following the procedure, it is known that the surgically created Fontan circulation often fails due to that high venous pressure required to drive pulmonary circulation. Long term mortality following the Fontan procedure can be as high as 29.1%, characterized by catastrophic failure of circulation and death. The expected event-free survival rate following the Fontan procedure at one, ten, and twenty-five years following the procedure is 80.1%, 74.8%, and 53.6%, respectively. A bi-modal age distribution has been observed in failing Fontan circulation. In early post-operative cases of failing Fontan circulation, the Fontan connection must be surgically taken down. In later post-operative cases, often the only remedy is heart transplantation.

Because of the above complications, in cases of failing Fontan circulation cavopulmonary assistance is often indicated, to assist or support movement of blood from the superior/inferior vena cava into pulmonary circulation, to decrease the central venous pressure required to provide the needed flow of blood to the lungs, and to reverse the pathophysiology associated with failing Fontan circulation. Attempts have been made to alleviate failing Fontan circulation by implanting a right ventricular assist device (RVAD). However, this requires a traumatic surgical intervention to implant the device, and also requires take-down of the Fontan connection to allow pump installation. Dual hemopumps have been evaluated to restore or assist failing Fontan circulation. However, in such cases using two hemopumps, two surgical site cannulations are required, which is unduly traumatic to a patient. Also, patient mobility is severely restricted when two pumps must be deployed by cannulation.

A number of smaller intravascularly deployable (at or near the site of Fontan anastomosis) pumps have been evaluated, but most also require two pumping mechanisms for deployment in the superior and inferior vena cava (above and below the surgically created Fontan connection) to move blood toward and into pulmonary circulation. Because of the dual pumps/dual cannulations required, such pumps cannot be made ambulatory, i.e. the patient must be substantially bedridden after deployment of the pumps. Single pump mechanisms have also been evaluated which can create the required flow of blood into the pulmonary artery, but have been found to be difficult to deploy and consistently maintain in position due to the need for precise placement at the Fontan anastomosis surgically created at the juncture of the superior/inferior vena cava and the pulmonary artery. For the latter reason, such single pumps cannot be made ambulatory and often require patient sedation to prevent pump displacement.

There is accordingly a need in the art for improved cavopulmonary assist devices (CPADs) to alleviate or remedy failing Fontan circulation. Such improved CPADs should require only a single cannulation, but should still promote satisfactory blood flow from both the superior and inferior vena cava into the pulmonary artery. Moreover, the CPADs should be relatively simple to deploy at the surgically created Fontan connection site.

### SUMMARY OF THE INVENTION

In accordance with the above-identified need in the art, in one aspect the present disclosure provides a dual-lumen cannula for cavopulmonary assistance, including a cannula body defining an infusing lumen and a draining lumen and a deployable barrier disposed on an exterior surface of the cannula body. Deploying the barrier within a vascular lumen substantially occludes fluid flow through the vascular lumen exterior to the dual lumen cannula, other than in an intravascular zone defined by the deployed barrier. In embodiments, the deployable barrier includes a first deployable wall spaced apart on the cannula body from a second deployable wall. The deployable walls may be configured as foldable membrane umbrellas or cones, typically having at least one end thereof attached to the cannula body exterior surface. The infusing lumen is in fluid communication with at least one outlet positioned in the cannula body between the first deployable wall and the second deployable wall whereby a fluid exiting the at least one outlet enters the intravascular zone. In an embodiment, the infusing lumen is in fluid communication with a first outlet and a second outlet positioned in the cannula body between the first deployable wall and the second deployable wall.

In another aspect, a percutaneous dual-lumen cannula based cavopulmonary assist device is provided, including a dual lumen cannula as described above and a pump for effecting fluid flow through the cannula infusing lumen and draining lumen.

In yet another aspect, a method for cavopulmonary assistance to alleviate failing Fontan circulation is provided. The method includes placing a percutaneous dual-lumen cannula based cavopulmonary assist device by advancing a dual lumen cannula as described above through a vascular lumen to a site of a Fontan anastomosis. The pump operatively connected to the dual lumen cannula effects fluid flow through the infusing lumen and draining lumen. The deployable barrier is then deployed.

On deployment of the deployable barrier, fluid flow through the vascular lumen exterior to the dual lumen cannula is substantially occluded but for an intravascular zone defined by the deployed barrier. The intravascular zone substantially bridges the site of the Fontan anastomosis whereby fluid exiting the infusing lumen increases fluid pressure within the intravascular zone, promoting fluid flow from the intravascular zone to into pulmonary circulation. To create the defined intravascular zone, a first deployable wall may be deployed at a site superior to the Fontan anastomosis, and a second deployable wall may be deployed at a site inferior to the Fontan anastomosis. In embodiments, the first and second deployable walls and infusing lumen outlets are positioned on the cannula body such that fluid exiting the infusing lumen causes deployment of the first and second deployable walls.

These and other embodiments, aspects, advantages, and features of the present invention will be set forth in the description which follows, and in part will become apparent to those of ordinary skill in the art by reference to the following description of the invention and referenced drawings or by practice of the invention. The aspects, advantages, and features of the invention are realized and attained by means of the instrumentalities, procedures, and combinations particularly pointed out in the appended claims. Various patent and non-patent citations are discussed herein. Unless otherwise indicated, any such citations are specifically incorporated by reference in their entirety into the present disclosure.

### BRIEF DESCRIPTION OF THE DRAWINGS

The accompanying drawings incorporated in and forming a part of the specification, illustrate several aspects of the present invention, and together with the description serve to explain the principles of the invention. In the drawings:

FIG. 1 depicts a human heart;

FIG. 2 depicts the heart of FIG. 1 after creation of a Fontan anastomosis to place the superior and inferior vena cava in fluid communication with the pulmonary artery;

FIG. 3 depicts a dual lumen cannula for cavopulmonary assistance according to the present disclosure;

FIGS. 4a and 4b depict an alternative embodiment of the dual lumen cannula of FIG. 3 showing an undeployed (FIG. 4) and deployed (FIG. 4b) barrier; and

FIGS. 5a and 5b depict an embodiment of the barrier of FIGS. 4a and 4b.

### DETAILED DESCRIPTION OF THE ILLUSTRATED EMBODIMENTS

In the following detailed description of the illustrated embodiments, reference is made to the accompanying drawings that form a part hereof, and in which is shown by way of illustration, specific embodiments in which the invention may be practiced. These embodiments are described in sufficient detail to enable those skilled in the art to practice the invention. Also, it is to be understood that other embodiments may be utilized and that process, reagent, software, and/or other changes may be made without departing from the scope of the present invention.

In the normal heart (see FIG. 1), the superior vena cava (SVC), inferior vena cava (IVC), and pulmonary artery (PA) are not in direct fluid communication. As is known in the art, in the Fontan procedure venous blood flow is surgically diverted from the SVC and IVC directly to the PA, bypassing the right ventricle of the heart. By this procedure, a surgically created junction (Fontan anastomosis F) is created to provide fluid communication from the superior and inferior vena cava to the pulmonary artery, and therefrom directly into pulmonary circulation (see FIG. 2). As summarized above, this Fontan circulation is prone to failure, requiring additional intervention to reverse or ameliorate failing Fontan circulation and associated pathologies.

In one aspect, the present disclosure provides a dual lumen cannula 10 (see FIG. 3) for cavapulmonary assistance to support or reverse failing Fontan circulation. The dual lumen cannula 10 includes a cannula body 12 defining an infusing lumen 14 and a draining lumen 16. The dual lumen cannula 10 further includes a deployable barrier 18 disposed on an exterior surface of the cannula body 12. As will be discussed in detail below, deploying the barrier 18 within a vascular lumen will substantially occlude fluid flow through the portion of the vascular lumen exterior to the dual lumen cannula 10 other than in an intravascular zone defined by the deployed barrier 18, thereby promoting blood flow into pulmonary circulation.

In an embodiment, the deployable barrier 18 includes a first deployable wall 20 and a second deployable wall 22 spaced apart on the cannula body 12. The deployable walls 20, 22 may be configured as any suitable structure which when deployed substantially occludes fluid flow through a vascular lumen into which the dual lumen cannula 10 is advanced, such as an inflatable balloon, a collapsible or foldable umbrella, a collapsible or foldable cone, etc. The infusing lumen 14 is in fluid communication with at least one outlet 24 positioned in the cannula body 12 between the first deployable wall 20 and the second deployable wall 22. As the skilled artisan will appreciate, fluid exiting the at least one outlet 24 enters the intravascular zone defined by the deployed barrier 18. The draining lumen is in fluid communication with a pair of inlets 26, 28. As shown, the inlets 26, 28 are each positioned in the cannula body 12 exterior of the deployable walls 20, 22 and the intravascular zone defined thereby.

In another embodiment shown in FIG. 4a, the infusing lumen 14 is in fluid communication with first and second outlets 30, 32 positioned in the cannula body 12 between the first deployable wall 20 and the second deployable wall 22. In the depicted embodiment, first outlet 30 is disposed adjacent to the first deployable wall 20, at or near an end 34 of the first deployable wall 20 which is permanently attached to an exterior surface of the cannula body 12. The second outlet 32 is positioned adjacent to the second deployable wall 22, at or near an end 44 of the second deployable wall 22 which is opposed to the end 36 that is permanently attached to an exterior surface of the cannula body 12. The advantages provided by this structure will be discussed in detail below.

In use, the dual lumen cannula 10 is introduced percutaneously using an atraumatic introducer (not shown) such as through the jugular vein of a patient (not shown), advanced into an intravascular space 38 in the superior vena cava, and therefrom is advanced to a site of a Fontan anastomosis F, i.e. to the surgically created anastomosis providing fluid communication between the patient's superior vena cava (SVC), inferior vena cava (IVC), right pulmonary artery (RPA) and left pulmonary artery (LPA) (see FIG. 4b). During placement of the dual lumen cannula 10, the undeployed deployable walls 20, 22 are releasably secured to cannula body 12 by any suitable means to prevent risk of undesired deployment of the walls prior to reaching the Fontan anastomosis site. In an embodiment, the deployable walls 20, 22 are folded on to the exterior surface of the cannula body 12 and secured thereto using a suitable water-soluble adhesive. The skilled artisan will appreciate that, on exposure of the water-soluble adhesive to an aqueous fluid such as blood, the adhesive will eventually dissolve and release deployable walls 20, 22 (except for connected ends 34, 36) to allow deployment thereof. A number of suitable non-toxic water/blood soluble adhesives are known in the art, including without intending any limitation dextrose.

As shown, the dual lumen cannula is advanced until the site of the Fontan anastomosis F is bridged, that is, until the first deployable wall 20 is positioned substantially superior to the Fontan anastomosis F, and the second deployable wall 22 is positioned substantially inferior to the Fontan anastomosis F. A number of imaging techniques for monitoring and verifying proper placement of the dual luman catheter 10 are known, including without intending any limitation X-ray fluoroscopy, radiography, intravascular ultrasound, and others.

A pump P is operatively connected to the dual lumen cannula 10 to create a fluid circuit, i.e. to introduce blood into (see arrows A) and to withdraw blood from (see arrows B) the inferior vena cava and superior vena cava. A number of suitable pumps are known in the art, such as in a non-limiting example the compact pump marketed under the trade name Centri-Mag (Levitronix, Waltham, Mass.). On deployment of the first and second deployable walls 20, 22, fluid flow from the SVC/IVC to pulmonary circulation exterior to the dual lumen cannula 12 is substantially occluded but for an area defined between the deployed walls 20, 22. As the skilled artisan will appreciate, fluid exiting via outlets 30, 32 into that area will create a zone of increased fluid pressure in the area between the deployed walls 20, 22, depicted in FIG. 4b as intravascular zone 40 (see shaded area). This intravascular zone 40 of increased fluid pressure will in turn promote fluid flow exiting the vena cava/intravascular zone 40 and draining therefrom into pulmonary circulation, i.e. into the right and left pulmonary arteries RPA, LPA (see arrows C). By this fluid flow, failing Fontan circulation can be supported and/or ameliorated.

An advantage of the depicted embodiment of the deployable walls 20, 22 will now be described. As discussed above, deployable walls 20, 22 could be provided in a number of configurations, such as asymmetric inflatable balloons, collapsible/foldable umbrellas or cones, etc. The embodiment depicted in FIG. 4b and shown in isolation in FIGS. 5a and 5b is a collapsible/foldable ultra-thin membrane umbrella or cone. As discussed above, outlets 30, 32 are positioned respectively at or near ends 34, 44 of deployable walls 20, 22, with at least ends 34, 36 of walls 20, 22 being attached to an exterior surface of the cannula body 12. In the embodiment depicted in FIG. 4b, the opposed ends 42, 44 of walls 20, 22 are not secured to the cannula body 12. However, it is possible that walls 20, 22 might overextend on deployment such that the desired occlusion of fluid flow through the intravascular space 38 is not obtained. Accordingly, optional retention means such as one or more flexible strings or wires 46 (see FIG. 5b) may be included to connect ends 42, 44 to cannula body 12 and prevent overextension on deployment of walls 20, 22.

On activation of pump P, fluid flow through infusing lumen 14 and draining lumen 16 is initiated (see FIG. 4b, arrows A and B). Of course, pump P may be a component of or otherwise associated with an oxygenator (not shown) to oxygenate blood removed from a patient via draining lumen 16, which is then returned to the patient by infusing lumen 14. As discussed above, infusing lumen 14 outlets 30, 32 are positioned on cannula body 12 respectively at or near ends 34, 44 of deployable walls 20, 22. Therefore, the flow of fluid exiting infusing lumen 14 via outlets 30, 32 will deploy the deployable walls 20, 22, such as by causing unfolding the folded umbrella/cone configuration described above and shown in FIG. 5b, and provide the desired occlusion of fluid flow through intravascular space 38. The increased fluid pressure in intravascular zone 40 will maintain the deployed configuration of walls 20, 22, and also promote fluid flow into pulmonary circulation (arrows C). Moreover, the relative position of outlets 30, 32 and walls 20, 22 at or near the ends 34, 44 of walls 20, 22 provides a good fluid flow at or near attached ends 34, 36, preventing or reducing areas of stagnant fluid flow underneath walls 20, 22. By this mechanism, the potential for thrombosis that might be encountered if outlets 30, 32 were positioned differently is avoided.

In the event of failure or shutdown of pump P, fluid flow through infusing lumen 14 and draining lumen 16 will cease. As the fluid flow caused by pump P decreases, the increased fluid pressure established in intravascular zone 40 will likewise decrease. As the skilled artisan will appreciate, the decreased fluid pressure in intravascular zone 40 will cause walls 20, 22 to at least partially collapse. This in turn will allow the previously occluded fluid flow through the SVC and IVC to at least partially resume, further collapsing the walls 20, 22. Accordingly, in the event of failure or inadvertent shutdown of pump P, fluid flow through the vena cava is not permanently occluded as would be the case with an inflated balloon, and circulatory collapse is avoided.

A number of significant advantages are thus realized by the dual lumen cannula 10 design set forth in the present disclosure. Only a single cannulation procedure is required to support failing Fontan circulation, improving patient comfort and mobility compared to prior art devices and methods requiring multiple cannulations. By the described design, on verification of proper placement of the dual lumen cannula, simply initiating fluid flow by a pump P to remove oxygen-depleted blood through draining lumen 16 and to supply oxygen-rich blood through infusing lumen 14 deploys the described deployable barrier 18 comprising deployable walls 20, 22. Thus, the need for more complex mechanical or surgical methods for deploying barrier 18 is avoided. In turn, the described positioning of infusing lumen 14 outlets reduces or eliminates areas of stagnation under the deployable walls 20, 22, reducing the risk of thrombosis. Deployment of barrier 18 creates a defined area 40 of increased fluid pressure at the site of a Fontan anastomosis, promoting flow of oxygen-enriched blood directly into pulmonary circulation. Thus, the requirement for high central venous blood pressure to promote blood flow from the vena cava directly to pulmonary circulation in a Fontan patient is also avoided. Still more, if pump P fails, the design of deployable walls 20, 22 allows them to collapse, at least partially restoring the prior Fontan circulation through the vena cava and into pulmonary circulation, thus preventing circulatory collapse until the pump can be replaced or repaired.

One of ordinary skill in the art will recognize that additional embodiments of the invention are also possible without departing from the teachings herein. This detailed description, and particularly the specific details of the exemplary embodiments, is given primarily for clarity of understanding, and no unnecessary limitations are to be imported, for modifications will become obvious to those skilled in the art upon reading this disclosure and may be made without departing from the spirit or scope of the invention. Relatively apparent modifications, of course, include combining the various features of one or more figures or examples with the features of one or more of other figures or examples.

What is claimed is:

1. A dual-lumen cannula for cavopulmonary assistance, comprising:
a cannula body defining an infusing lumen and a draining lumen; and
a deployable barrier disposed on an exterior surface of the cannula body;
wherein deploying the barrier within a vascular lumen substantially occludes fluid flow through a portion of the vascular lumen exterior to the cannula body other than an intravascular zone defined by the deployed barrier.

2. The dual-lumen cannula of claim 1, wherein the deployable barrier includes a first deployable wall spaced apart from a second deployable wall.

3. The dual-lumen cannula of claim 2, wherein the first deployable wall and second deployable wall are foldable membrane umbrellas or cones having at least one end thereof attached to the exterior surface.

4. The dual-lumen cannula of claim 2, wherein the infusing lumen is in fluid communication with at least one outlet positioned in the cannula body between the first deployable wall and the second deployable wall whereby a fluid exiting the at least one outlet enters the intravascular zone.

5. The dual-lumen cannula of claim 4, wherein the infusing lumen is in fluid communication with a first outlet and a second outlet positioned in the cannula body between the first deployable wall and the second deployable wall.

6. The dual-lumen cannula of claim 2, wherein the draining lumen is in fluid communication with a first inlet and a second inlet each positioned in the cannula body exterior of the first deployable wall and the second deployable wall.

7. A percutaneous dual-lumen cannula based cavopulmonary assist device, comprising:
a dual-lumen cannula including a body defining an infusing lumen and a draining lumen, further including a deployable barrier disposed on an exterior surface of the cannula body whereby deploying the barrier within a vascular lumen substantially occludes fluid flow through a portion of the vascular lumen exterior to the cannula body other than an intravascular zone defined by the deployed barrier; and
a pump for effecting fluid flow through the infusing lumen and the draining lumen.

8. The cavopulmonary assist device of claim 7, wherein the deployable barrier includes a first deployable wall spaced apart from a second deployable wall.

9. The cavopulmonary assist device of claim 8, wherein the first deployable wall and second deployable wall are foldable membrane umbrellas or cones having at least one end thereof attached to the exterior surface.

10. The cavopulmonary assist device of claim 8, wherein the infusing lumen is in fluid communication with at least one outlet positioned in the cannula body between the first deployable wall and the second deployable wall whereby a fluid exiting the at least one outlet enters the intravascular zone.

11. The cavopulmonary assist device of claim 10, wherein the infusing lumen is in fluid communication with a first outlet and a second outlet positioned in the cannula body between the first and second deployable walls.

12. The cavopulmonary assist device of claim 8, wherein the draining lumen is in fluid communication with a first inlet and a second inlet each positioned in the cannula body exterior of the first deployable wall and the second deployable wall.

13. A method for providing cavopulmonary assistance to failing Fontan circulation, comprising:
advancing a percutaneous dual-lumen cannula through a vascular lumen to a site of a Fontan anastomosis, the dual-lumen cannula comprising a body defining an infusing lumen and a draining lumen and a deployable barrier disposed on an exterior surface of the cannula body;
causing a pump operatively connected to the dual-lumen cannula to effect a fluid flow through the infusing lumen and the draining lumen; and
deploying the deployable barrier to substantially occlude fluid flow through a portion of the vascular lumen exterior to the cannula body other than an intravascular zone defined by the deployed barrier, said intravascular zone substantially bridging the site of the Fontan anastomosis;
whereby a fluid exiting the infusing lumen increases a fluid pressure within the intravascular zone and promotes a fluid flow from the intravascular zone into a pulmonary circulation.

14. The method of claim 13, wherein deploying the deployable barrier includes:
deploying a first deployable wall in the vascular lumen at a site superior to the Fontan anastomosis; and
deploying a second deployable wall in the vascular lumen at a site inferior to the Fontan anastomosis.

15. The method of claim 14, wherein the first deployable wall and second deployable wall are foldable membrane umbrellas or cones having at least one end thereof attached to the exterior surface.

16. The method of claim 13, including effecting the fluid flow from the infusing lumen into the intravascular zone by at least one outlet in fluid communication with the infusing lumen and positioned between the first deployable wall and the second deployable wall.

17. The method of claim 16, including effecting the fluid flow from the infusing lumen into the intravascular zone by a first outlet in fluid communication with the infusing lumen and positioned below the first deployable wall and a second outlet in fluid communication with the infusing lumen and positioned above the second deployable wall.

18. The method of claim 13, including effecting the fluid flow through the draining lumen by a first inlet in fluid communication with the draining lumen and positioned above the first deployable wall and a second inlet in fluid communication with the draining lumen and positioned below the second deployable wall.

19. The method of claim 13, wherein the fluid exiting the infusing lumen deploys the deployable barrier.
